(19) United States
(12) Patent Application Publication
(10) Pub. No.: US 2011/0184372 A1
(43) Pub. Date: Jul. 28, 2011

(54) ABSORBENT GARMENT PROVIDED WITH SIDE PANELS OR BELT MEMBERS

(76) Inventors: Hanna Esping Östlin, Pixbo (SE); Birgitta Karlsson, Molnlycke (SE); John Wreland, Huskvarna (SE)

(21) Appl. No.: 12/809,117

(22) PCT Filed: Dec. 21, 2007
(86) PCT No.: PCT/SE07/01165

§ 371 Date: Apr. 4, 2011

(51) Int. Cl. A61F 13/64

(52) U.S. Cl. 604/392

(57) ABSTRACT

An absorbent garment such as a diaper or an incontinence guard provided with a pair of laterally extending side panels or at least one belt member. At least one of the side panels or belt members includes at least two elastic regions, a first and a second elastic region, each extending over only a part of the length of and over only a part of the distance between the upper and lower edges of the respective side panel or belt member. The first elastic region has a main part of its surface area on the upper part of the respective side panel or belt member, and the second elastic region has a main part of its surface area on the lower part of the respective side panel or belt member.

### TECHNICAL FIELD

The present invention refers to an absorbent garment such as a diaper or an incontinence guard provided with elastic side panels or belt members as part of the fastening means for attaching the garment around the waist of the wearer.

### BACKGROUND OF THE INVENTION

Absorbent garments having elastic side panels extending laterally outwardly from a waist section of the garment are well known. Side panels are used in order to attach one body panel, usually the rear body panel, to the opposite body panel, usually the front body panel. For this purpose the side panels are provided with releasable fastening means, preferably in the form of mechanical fasteners of the hook and loop type (e.g. Velcro®), or adhesive tape fasteners. At least part of the side panels may be of an elastic material in order to provide an improved fit around the waist of the wearer.

One example of an absorbent garment with elastic side panels is shown in EP-A-1 350 493. Each side panel has an elastic section which extends over only a part of the distance between the upper and lower edges of the side panel, wherein material costs are reduced without unduly affecting the functionality of the garment.

Another example is JP-2006102278 which discloses a diaper with side panels having an upper region with more elasticity than the lower region.

A further example is FR-A-2 888 490 disclosing a diaper having side panels with first elastic elements extending along the upper edge of the side panels and second elastic members extending diagonally across the side panels.

So called belted absorbent garments comprise a front body panel adapted to be applied over the stomach of the wearer, a back body panel adapted to be applied over the back of the wearer and a crotch portion adapted to extend over the crotch of the wearer between the legs. The garment further comprises either one laterally extending belt or a pair of opposed laterally extending belt members usually attached to the back body panel at the waist portion thereof. These belt portions are adapted to be wrapped around the waist of the wearer of the garment and fastened together by means of first fastening means, such as an adhesive tape or mechanical fastening means, for example a hook-and-loop fastener. The front body panel is then passed between the legs of the wearer and fastened to the outside of the belt members by means of second fastening means provided at the waist portion of the front panel. The garment will then assume a pant-like shape. Alternatively the belt portions are arranged at the front body panel and the second fastening means at the back body panel. Since often the front and back body panels are more or less identical as to shape and size the wearer or caretaker may apply the article after individual preference, so that the front body panel may be used as back body panel and vice versa.

The belt provides for improved possibilities to adjust the fit of the absorbent garment. It further simplifies the change of the garment especially when the wearer is standing up.

Examples of belted absorbent garments are found in for example EP-A-0 287 388, EP-A-0 409 307, EP-A-0 605 012 and FR-A-2 586 558.

It is also known to make part of the belt elastic in order to further improve comfort and fit. JP-B-3471999 discloses a belted diaper wherein the belt portions are attached to the lateral side edges of the back panel and have a stretchable part at the respective base end thereof, which allows the belt portions to be extended along their length direction. The back panel of the diaper is further provided with an elastic portion (waist elastics).

EP-A-1 110 529 discloses a belted diaper wherein the belt members are interconnected by a waist band which is attached by adhesive to the external side of the back panel of the diaper. One portion close to the distal end of the belt members may be elastic.

The side panels or belts may either be permanently attached to the chassis of the garment and discarded together with the garment after use, or be releasably attached to the chassis and thus be reusable. Examples of absorbent articles having reusable belts releasably attached thereto are found in EP-B-1 011 583 and in U.S. Pat. No. 5,135,522,

There is however still a need for absorbent garments having side panels or belt members which have an improved fit around the waist for wearers of different sizes and body shapes, since some wearers have their largest dimensions over the hip area and other over the stomach. An improved adaptation of the belt dimension to the wearers body also when the wearer is moving is further desired. Since elastic materials in general are more expensive than inelastic materials, it is also a desire to keep the amount of elastic material as low as possible and only in locations where it fulfils a useful function.

### OBJECT AND MOST IMPORTANT FEATURES OF THE INVENTION

The present invention aims at providing an absorbent garment with elastic side panels or belt members having improved comfort and fit for different sizes and body shapes of wearers and which adapts well to the wearers body also when the wearer is moving. This has according to the present invention been accomplished by the fact that at least one of the side panels or belt members comprises at least two elastic regions, a first and a second elastic region, each extending over only a part of the length of the respective side panel or belt member and over only a part of the distance between the upper and lower edges of the respective side panel or belt member, the first elastic region having a main part of its surface area on the upper part of the respective side panel or belt member, so as to allow elastic expansion of the upper edge in the area of the first elastic region, and there is a portion of inelastic material between the first elastic region and the lower edge of the side panel or belt member, and the second elastic region has a main part of its surface area on the lower part of the respective side panel or belt member, so as to allow elastic expansion of the lower edge in the area of the second elastic region, and there is a portion of inelastic material between the second elastic region and the upper edge of the side panel or belt member.

In one embodiment the elastic regions extend over from 20 to 80%, preferably over from 30 to 70% and more preferably from 45 to 55% of the distance between the upper and lower edges of the respective side panel or belt member.

In a further aspect the elastic regions each has a length in the lateral direction of the side panel or belt member and a height in a direction transverse thereto, wherein the height of each elastic region is at least 2 cm, preferably at least 3 cm.

According to one embodiment of the invention said side panel or belt member has a height in a direction transverse to the lateral direction of between 8 and 40 cm, preferably between 8 and 35 cm.

In a further embodiment said elastic regions each has a length in the lateral direction of the side panel or belt member of between 1.5 and 20 cm, preferably between 2 and 15 cm and more preferably between 2 and 10 cm.

In one aspect of the invention at least one of said side panels or belt members comprises at least three elastic regions, first, second and third elastic regions, wherein also the third elastic region extends over only a part of the length of the respective side panel or belt member and over only a part of the distance between the upper and lower edge of the respective side panel or belt member and wherein the third elastic region has a main part of its surface area on the upper or lower part of the respective side panel or belt member, so as to allow elastic expansion of the upper edge or lower edge, respectively, in the area of the third elastic region, and there is a portion of inelastic material also between the third elastic region and the lower or upper edge respectively of the side panel or belt member. According to one embodiment the third elastic zone is located on the upper part of the respective side panel or belt member.

In a further embodiment at least one of said side panels or belt members comprises at least four elastic regions, first, second, third and fourth elastic regions, wherein also the fourth elastic region extends over only a part of the length of the respective side panel or belt member and over only a part of the distance between the upper and lower edge of the respective side panel or belt member and wherein the fourth elastic region has a main part of its surface area on the upper or lower part of the respective side panel or belt member, so as to allow elastic expansion of the upper edge or lower edge, respectively, in the area of the fourth elastic region and there is a portion of inelastic material also between the fourth elastic region and the lower or upper edge respectively of the side panel or belt member. According to one embodiment two elastic zones are located on the lower part and two elastic zones are located on the upper part of the respective side panel or belt member.

In a still further embodiment at least one of said side panels or belt members comprises at least five and preferably at least six elastic regions, each extending over only a part of the length of the respective side panel or belt member and over only a part of the distance between the upper and lower edge of the respective side panel or belt member so as to allow elastic expansion of the upper edge or lower edge, respectively, in the area of each elastic region. According to one embodiment at least three of said elastic regions are located on the upper part of the respective side panel or belt member.

According to one aspect of the invention both side panels or belt members comprise said elastic regions referred to above. The number of elastic regions on the two side panels or belt members may be the same or different.

The elastic regions may comprise an elastic film or an elastic laminate. Alternatively it comprises one or more elastic threads or strips contractably affixed between web materials.

In one embodiment at least a substantial part of the side panels or belt members comprises a material that can be made elastic by activation and the elastic regions are regions that have been activated so as to become elastic.

In one embodiment of the invention the elastic region of the respective belt member is provided with a contrasting colour, pattern or marking so as to be distinguishable from the rest of the belt member, or alternatively that said rest of the belt member is provided with a contrasting colour, pattern or marking so as to be distinguishable from said elastic region.

### BRIEF DESCRIPTION OF THE DRAWINGS

The invention will in the following be closer described with reference to some embodiments shown in the accompanying drawings.

FIG. 1 shows a plan view of a diaper as seen from the wearer facing side having elastic side panels according to the invention.

FIG. 2 shows a plan view of a belted diaper as seen from the wearer facing side according to one embodiment of the invention.

FIGS. 3 and 4 are a plan views on a larger scale of a belt according to further embodiments of the invention.

FIG. 5a shows an embodiment of an absorbent article comprising a releasable belt in a view in which the absorbent unit is separated from the belt.

FIG. 5b shows the article of FIG. 5a in a position in which the absorbent unit is attached to the belt.

### DESCRIPTION OF PREFERRED EMBODIMENTS

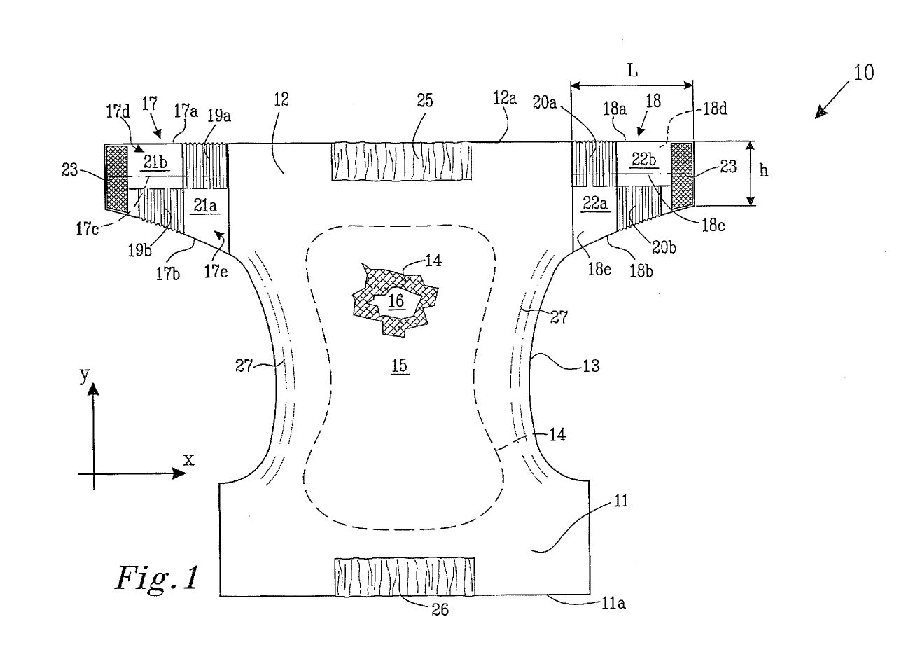

FIG. 1 of the drawings shows an embodiment of an absorbent garment in the form of a diaper, a sanitary article or incontinence guard 10. The term “absorbent garment” is refers to an absorbent article which is intended to be worn around the waist of the wearer like a pair of pants. The absorbent garment 10 comprises a first body panel 11, which in the embodiment shown in the drawings is the part of the garment that in use is intended to extend over the stomach and front hip area of the wearer. The garment also comprises a second body panel 12, which in the shown embodiment is the part of the garment that in use is intended to extend over back and the rear hip area of the wearer. Each of said first and second body panels 11 and 12 has a waist portion 11a and 12a respectively. It is understood that in other embodiments the first body panel 11 may be the back body panel and the second body panel 12 may be the front body panel.

The crotch portion 13 of a garment 10 is the part of the garment that in use is intended to extend through the wearer's crotch area, between the legs. An absorbent core 14 is disposed in the crotch portion 13 and extends into the first and second body panels 11 and 12. The absorbent core 14 is disposed between an inner coversheet 15 and an outer coversheet 16. The garment has a longitudinal direction, y, and a lateral direction, x.

The term “inner coversheet” refers to a liquid permeable material sheet forming the inner cover of the absorbent garment and which in use is placed in direct contact with the skin of the wearer. The inner coversheet can comprise a nonwoven material, e.g. spunbond, meltblown, carded, hydroentangled, wetlaid etc. Suitable nonwoven materials can be composed of man-made fibres, such as polyester, polyethylene, polypropylene, viscose, rayon etc. or natural fibers, such as wood pulp or cotton fibres, or from a mixture of natural and man-made fibres. The inner coversheet material may further be composed of tow fibres, which may be bonded to each other in a bonding pattern, as e.g. disclosed in EP-A-1 035 818. Further examples of inner coversheet materials are porous foams, apertured plastic films, laminates of nonwoven materials and apertured plastic films etc. The materials suited as inner coversheet materials should be soft and non-irritating to the skin and be readily penetrated by body fluid, e.g. urine or menstrual fluid. The inner coversheet may further be different in different parts of the absorbent garment.

The “outer coversheet” refers to a material forming the outer cover of the absorbent garment. The outer coversheet may be the same or different in different parts of the absorbent garment. At least in the area of the absorbent core the outer coversheet comprises a liquid impervious material in the form of a thin plastic film, e.g. a polyethylene or polypropylene film, a nonwoven material coated with a liquid impervious material, a hydrophobic nonwoven material, which resists liquid penetration, or a laminate of a plastic film and a nonwoven material. The outer coversheet material may be breathable so as to allow vapour to escape from the absorbent core, while still preventing liquids from passing there through. Examples of breathable outer coversheet materials are porous polymeric films, nonwoven laminates of spunbond and meltblown layers and laminates of porous polymeric films and nonwoven materials. Preferably, the outer coversheet comprises a nonwoven material on at least the undergarment-facing surface thereof.

The “absorbent core” is the absorbent structure disposed between the two coversheets of the absorbent garment in at least the crotch region thereof. The absorbent core can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as superabsorbent materials can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form which is suitable for use in absorbent composites including particles, fibres, flakes, spheres, and the like.

A high liquid storage capacity is provided by the use of high amounts of superabsorbent material. For an absorbent core comprising a matrix of hydrophilic fibres, such as cellulosic fibres, and superabsorbent material, the proportion of superabsorbent material is preferably between 10 and 90% by weight, more preferably between 30 and 70% by weight.

It is conventional for absorbent garments to have absorbent cores comprising layers of different properties with respect to liquid receiving capacity, liquid distribution capacity and storage capacity. The absorbent bodies, which are common in for example baby diapers and incontinence guards, often comprise a compressed, mixed or layered structure of cellulosic fluff pulp and superabsorbent polymers. The size and absorbent capacity of the absorbent core may be varied to suit different uses, such as infants or adult incontinent persons.

The absorbent core may further include an acquisition distribution layer placed on top of the primary absorbent body, which is adapted to quickly receive and temporarily store discharged liquid before it is absorbed by the primary absorbent core. Such acquisition distribution layers are well known in the art and may be composed of porous fibrous wadding or foam materials.

On each side of the waist portion 12a of the second body panel 12 a side panel 17 and 18 respectively is located. The side panels 17, 18 are preferably substantially identical. The side panels 17, 18 have a length, L, in lateral direction, x, of the garment and a height, H, in the longitudinal direction, y, of the garment, wherein the height H may vary along the length of the side panels, as is the case in for example FIG. 1.

The side panels 17, 18 each has an upper edge 17a, 18a a lower edge 17b, 18b and a lateral centre line 17c, 18c. The upper edge 17a, 18a is in the shown embodiment substantially in line with the upper edge of the second body panel 12. The lower edge 17b, 18b of each side panel 17, 18 has in this embodiment a curved shape. The lateral centre line 17c, 18c for a side panel having a varying height H, as shown in FIG. 1, is defined as a line which extends in the lateral direction, x, of the garment, and which passes through the longitudinal centre, as seen in the y direction, of the part of the side panel having the smallest height, H, in this case the outermost portion of the side panel 17, 18. This lateral centre line 17c, 18c divides the side panels in an upper and a lower part, 17d, 18d and 17e, 18e.

Each side panel 17, 18 has at the respective outer edge portion a first fastening means 23, for example a mechanical fastening means in the form of a hook or loop member, intended to cooperate with a complementary mechanical fastening means on the outside of the first body panel 11. The entire external surface of the outer cover 16 of the first body panel may comprise a nonwoven material which functions as a loop material or a separate strip of loop material may be attached to the external surface of the first body panel 11.

Further examples of mechanical fasteners are button and holes or button loops, snap fasteners and the like. The buttons can either be fastened to the belt or to the garment.

A “hook-and-loop fastener” refers to complementary fastening means having a “hook” portion and a “loop” portion and which are refastenable. The term “hook” as used herein refers to any element capable of engaging another element, the so called “loop” portion. The term “hook” is not limited to only “hooks” in its normal sense, but rather encompasses any form of engaging elements, whether unidirectional or bi-directional. The term “loop” is likewise not limited to “loops” in its normal sense, but also encompasses any structure capable of engaging with a “hook” fastener. Examples of “loop” materials are fibrous structures, like nonwoven materials. Hook-and-loop fasteners are for example available from Velcro, USA.

Alternatively the first fastening means 23 is an adhesive fastening means such as a tape tab, wherein at least part of the external surface of the outer cover 16 of the first body panel 11 is of a material to which the tape can adhere.

Each side panel 17, 18 further comprises two elastic regions 19a, 19b and 20a, 20b respectively, each extending over only a part of the length, L, of the respective side panel 17, 18 and over only a part of the distance between the upper and lower edges 17a and 17b and 18 a an 18b of the respective side panel. The first elastic region 19a, 20a has a main part of its surface area on the upper part 17d, 18d of the respective side panel 17, 18 and the second elastic region 19b, 20b has a main part of its surface area on the lower part 17e, 18e of the side panel. The rest of the side panels 17, 18 are of an inelastic material, wherein there is a portion 21a, 22a of inelastic material between the first elastic region 19a, 20a and the lower edge 17b, 18b of the respective side panel 17 and 18, and there is a portion 21b, 22b of inelastic material between the second elastic region 19b, 20b and the upper edge 17a, 18a of the respective side panel.

The term “elastic” in this respect refers to a material having an elastic elongation of at least 30% as measured in the Elasticity test as described below.

The term “inelastic” in this respect refers to a material having an elastic elongation of not more than 20% as measured in the Elasticity test as described below.

Said elastic regions may be in the form of an elastic web material such as an elastic film, an elastic nonwoven, an elastic laminate or the like. The elastic laminate may be a laminate between two or more nonwoven layers, two or more film layers or a combination of film and nonwoven layers.

Examples of elastic laminates suitable for forming said elastic regions are any elastic laminate known in the art. One group of elastic laminates are so called “stretch-bonded” laminates, in which the elastic layer is stretched in at least one direction before laminating it with one or more inelastic layers. After the tension is removed from the elastic layer it can freely retract to its untensioned state, and the inelastic layer(s) laminated thereto become gathered, giving a three-dimensional puckering.

Another group of elastic laminates are so called “neck bonded” laminates, which refer to laminates in which an elastic material is bonded to a non-elastic material while the non-elastic member is extended under conditions reducing its width or necked. “Neck bonded laminate” refers to a composite material having at least two layers in which one layer is a necked, non-elastic layer and the other layer is an elastic layer. The layers are joined together when the non-elastic layer is in an extended condition.

A further group of elastic laminates are disclosed in for example WO03/047488, in which inelastic nonwoven layers are laminated to an elastic film layer, and the laminate is stretched above the point of failure of the nonwoven materials, so that the inelastic layers break.

Further examples of elastic laminates are described in EP-B-0 646 062, WO 98/29251, WO 03/000165 and U.S. Pat. No. 5,226,992. Examples of commercially available elastic laminates are Fabriflex 306™ from Tredegar and PK 6358™ from Nordenia.

In an alternative embodiment the elastic regions comprise one or more elastic threads or strips contractably affixed between material sheets, for example nonwoven materials.

In a still further embodiment the side panels 17, 18, or at least substantial parts thereof, are of a material that can be made elastic by activation and the elastic regions 19a, b and 20a, b are regions that have been activated, for example by heating, so as to become elastic.

A waist elastic member 25 extends in transverse direction, x, along at least part of the waist portion 12a of the second body panel 12. The waist elastic member may be an elastic web material such as an elastic laminate, an elastic film, an elastic nonwoven or the like contractably attached between the inner and outer coversheets 15 and 16, to the external side of the outer coversheet or to the wearer facing side of the inner coversheet. Alternatively it comprises two or more elastic threads or strips contractably affixed between the outer and inner coversheets.

A waist elastic member 26 extends in transverse direction, x, also along at least part of the waist portion 11a of the first body panel 11.

The waist elastic members 25, 26, where present, allow an extension of the waist area to allow the users's movements, when standing and sitting for example, to be taken into account whilst still providing a comfortable and well-fitting waist portion. The waist elastics are preferably located outside the longitudinal limits of the absorbent core 14. It is understood that the garment may lack waist elastics or have waist elastics only in the front or the back body panel 11 or 12.

The leg openings may be elasticized, said elastification is usually accomplished by a plurality of elastic members 27, such as elastic threads, which are contractably affixed between the outer and inner coversheets. The garment may also be provided by so called barrier cuffs (not shown), in order to provide an improved security against leakage. These barrier cuffs may in some instance replace leg elastics.

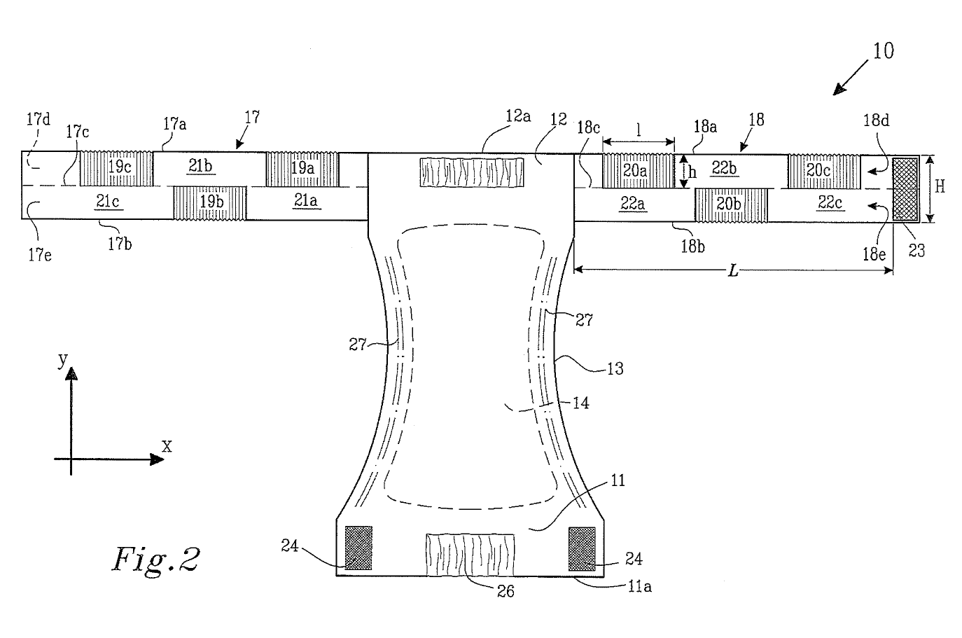

FIG. 2 shows a belted diaper or incontinence garment in which a pair of belt members 17, 18 are attached to the waist portion of second body panel 12 and are intended to be wrapped around the waist of the wearer of the garment and fastened together by means of first fastening means 23, for example a mechanical fastener, especially a hook fastener of a hook-and-loop fastening means. The external surface of the opposite belt member, especially if this comprises a fibrous nonwoven, may function as a loop material. Further examples of fastening means are listed above with respect to the embodiment shown in FIG. 1.

In an alternative embodiment (not shown) the absorbent article has only one belt member attached to one side edge of the waist region and is intended to be wrapped around the waist of the wearer and be fastened to the opposite side of the waist region.

Second fastening means 24 are provided at the lateral edges of the waist portion 12a of the front panel 12 as illustrated in FIG. 1. These second fastening means 24 are of the kind mentioned above with respect to the first fastening means 23 and are intended to be fastened to the external surface of the belt members 17, 18, after these have been wrapped around and fastened together around the user's waist.

Each belt member has a length, L, and a height, H, wherein the height may vary in the length direction of the belt member or be constant. However belt members usually have a substantially constant height H along the main part of their length and for such belts the height, H, is usually from 5 to 40 cm, preferably from 8 to 30 cm, at least along those parts having the substantially constant height.

Each belt member 17, 18 has an upper edge 17a, 18a, a lower edge 17b, 18b and a lateral centre line 17c, 18c. The upper edge 17a, 18a is in the shown embodiment substantially in line with the upper edge of the second body panel 12 and the lower edge 17b is substantially parallel therewith. The lateral centre line for a belt member having a varying height, H, along its length is as defined above with respect to the side panels 17, 18 in FIG. 1. This lateral centre line 17c, 18c divides the side panels in an upper and a lower part, 17d, 18e and 17e, 18e respectively.

Each belt member 17, 18 comprises three elastic regions 19a-c and 20a-c respectively, each extending over only a part of the length, L, of the respective belt member 17, 18 and over only a part of the distance between the upper and lower edges 17a, 18a and 17b, 18b of the respective belt member. The first elastic region 19a, 20a has a main part of its surface area on the upper part 17d, 18d of the respective belt member 17, 18, the second elastic region 19b, 20b has a main part of its surface area on the lower part 17e, 18e of the belt member and the third elastic region 19c, 20c has a main part of its surface area on the upper part 17d, 18d of the respective belt member 17, 18.

The rest of the belt members 17,18 outside the elastic regions 19, 20 are of an inelastic material, wherein there is a portion 21a, 22a and 21c, 22c of inelastic material between the first and third elastic regions 19a, 20a and 19c, 20c and the lower edge 17b, 18b of the respective belt member, and there is a portion 21b, 22b of inelastic material between the second elastic region 19b, 20b and the upper edge 17a, 18a of the respective belt member.

The elastic regions 19a-c, 20a-c have an elasticity as specified above with respect to the embodiment of FIG. 1 and examples of suitable materials for the elastic regions are the same as those listed above.

The belted garment of FIG. 2 is further similar to the absorbent garment described with respect to FIG. 1 and may optionally comprise components like leg elastics 27 and waist elastics 25 and 26 for example.

The rest of the belt members outside the elastic regions 19a-c, 20a-c is of a material that is substantially inelastic in the same manner as specified above in relation to the embodiment of FIG. 1.

A suitable nonwoven material forming at least part of the belt members outside the elastic regions is a spunbond material of e.g. polypropylene or polyethylene fibres. Conjugate fibres may also be used. Another suitable nonwoven material is formed from a carded thermobonded material of e.g. polypropylene, polyester or conjugate fibres.

The external surface of the belt members, or at least substantial parts thereof, should be adapted to function as a reception surface for the first and second fastening means 23 and 24. In case the fastening means are hook fasteners a nonwoven material may be used as external surface of the belt members. In case the fastening means 23 and 24 are adhesive tape tabs, a plastic film is suitable as external surface.

The inside of the belt members 17, 18 intended to be in direct contact with the skin of the wearer should be of a soft nonwoven material. A laminate having an outer nonwoven layer adapted as a loop material for a hook fastener and an inner soft and non-irritating nonwoven layer is an example of a suitable material for the belt members, at least in the regions outside the elastic regions.

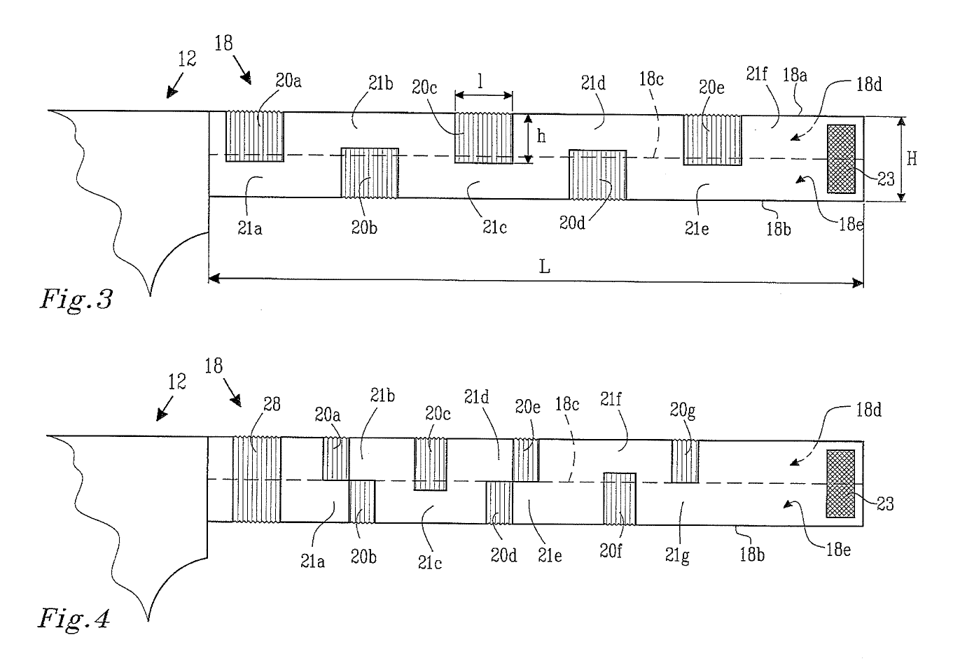

In the embodiment shown in FIG. 3, which shows only one belt member 18, said belt member 18 has four elastic regions 20a-d wherein two elastic regions 20a and c are located on the upper portion 18d and two elastic regions 20b and d on the lower portion 18e of the belt member.

In the embodiment shown in FIG. 4 the belt member 18 has six elastic regions 20a-f, wherein three elastic regions 20a, c and e are located on the upper portion 18d and three elastic regions 20b, d and f on the lower portion 18e of the belt member. In addition the belt member 18 comprises close to its proximal end adjacent the first body panel 11, a further elastic region 28, which extends the entire height, H, of the belt member 18. Said further elastic region 28 may be of the same kind as specified for the elastic regions 20.

The dimensions of the elastic regions 19, 20 may vary depending for example on the dimensions of the side panel or belt member 17, 18. Thus a baby diaper and an incontinence guard for adults have very different dimensions. The height, h, of each region is according to one aspect of the invention from 20 to 80% and preferably from 30 to 70% of the distance between the upper and lower edges 17 a and b, 18a and b respectively (or height H) of the respective side panel or belt member 17, 18 at the location of the respective elastic region. It is preferred that the elastic regions 19, 20 have a height, h, of at least 2 cm.

In a further aspect the elastic regions 19, 20 each has a length, l, in the lateral direction, x, of the side panel or belt member 17, 18 from 1.5 to 20 cm, preferably from 2 to 15 cm and more preferably from 2 to 10 cm.

The dimensions of the elastic regions do not have to be the same on one side panel or belt member, so that one or more elastic regions may be larger than the other(s). They may also be arranged with the same or different spacing between them along the length of the respective side panel or belt member.

The number of elastic regions 19, 20 on each side panel or belt member 17, 18 may vary, but should be at least two, wherein one elastic region 19a, 20a is located on the upper part 17d, 18d and one the lower part of the respective side panel or belt member. In further embodiments three, four, five, six or more elastic regions are located on the respective side panel or belt member. In all these embodiments at least one elastic region should be located on the opposite part (upper or lower) of the side panel or belt member as the other elastic regions. Thus if four elastic regions are present, three may be located on the upper part 17d, 18d and one on the lower part 17e, 18e of the side panel or belt member or vice versa, or two may be located on the upper part 17d, 18d and two on the lower part 17e, 18d.

It is also conceivable that different numbers of elastic regions 19, 20 are present on the two opposite side panels or belt members or that only one side panel or belt member 17, 18 is provided with elastic regions 19, 20. In addition the belt member 18 comprises close to its proximal end adjacent the first body panel 11, a further elastic region 28, which extends the entire height, H, of the belt member 18.

In the embodiments shown in FIGS. 2-4 the belt members 17, 18 are permanently attached to the second body panel 12, for example by gluing, heat bonding, ultrasonic welding or the like, which means that they are not intended to be released from the rest of the absorbent garment. The belt members 17, 18 may be two separate members attached to opposite sides of the second body panel or may be interconnected by an intermediate portion extending across and attached to the waist portion 12a of the second body panel 12.

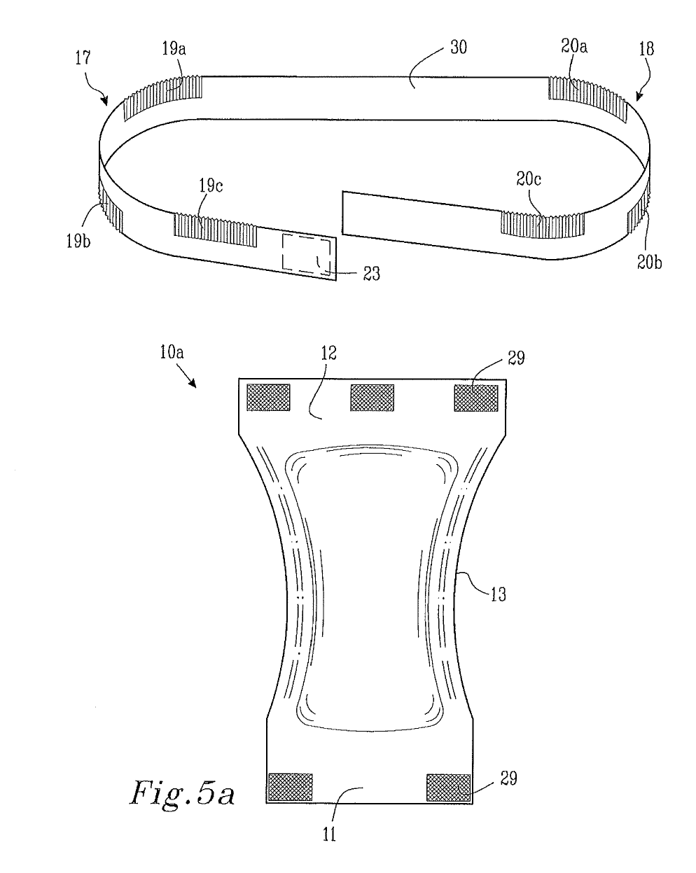

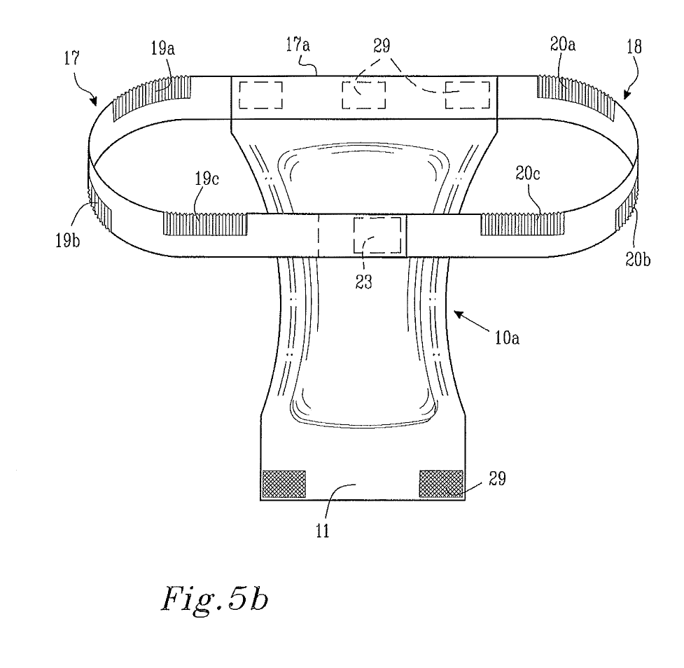

In an alternative embodiment shown in FIG. 5a and b an embodiment is shown in which a waist belt comprising belt members 17, 18 is releasably attachable to an absorbent unit 10a for forming an absorbent garment 10. After use of the article the absorbent unit 10a is discarded, while the waist belt is released from the absorbent unit 10a and may be reused. The belt members 17, 18 are interconnected by an intermediate member 30, which may be of the same or of a different material than the belt members 17, 18.

The absorbent unit 10a has a front body panel 11, a back body panel 12 and a crotch portion 13 and may optionally comprise features like leg elastics, waist elastics etc. as described above with respect to the embodiments of FIGS. 1 and 2. The waist areas of the front and back body panels are provided with fasteners 29 for fastening the absorbent unit 10a to the intermediate portion 30 of the belt. The fasteners 29 may either be located on the wearer facing or the garment facing side of the absorbent unit 10a. In one embodiment the fasteners 29 are located on the wearer facing side of the absorbent unit 10a, so that they will attach to the garment facing side of the belt, as is shown in FIGS. 5a and b.

The fasteners 29 may be of the same kind as the fasteners 23 and 24 in the embodiments of FIGS. 1 and 2. The entire garment facing side of the belt 17 may be covered with a material to which the fasteners 29 can attach. If the fasteners 29 shall be attached to the wearer facing side of the intermediate part 30 of the belt, the wearer facing surface of at least the intermediate part 30 of the belt should function as an attachment surface for the fasteners 29. Alternatively, discrete members of complementary fastening means to which the fasteners can attach are arranged on the intermediate part 30 of the belt. In a still further embodiment fasteners are arranged on the belt and complementary fastening means or attachment surfaces are provided at the waist portions of the absorbent unit 10a.

The belt portions 17 and 18 of the belt in FIGS. 5a and b is each provided with three elastic regions 19a-c and 20a-c in a corresponding manner as described above with respect the previous embodiments. Also for a reusable belt the number and dimensions of these elastic regions may vary.

In one embodiment of the invention the elastic regions 19, 20 of the belt members 17, 18 are indicated by a contrasting colour, pattern or marking, so as to be readily distinguishable to the user or caregiver for indicating that this region should not be used as an attachment surface for the fastening means 23, 24, 29 as the effectiveness of the fastening means is decreased if attached to an elastic region, since the elastic region may expand and/or contract and thereby weaken the bond between the fastening means an the elastic region. Alternatively the non-elastic portions of the belt members 17, 18 are indicated with said contrasting colour, pattern or marking for indicating that this region should be used as attachment surface.

An important advantage of the alternating arrangement of the elastic regions 19, 20 on the upper and lower parts 17d, 18d and 17e, 18e of the belt members 17, 18 is an improved comfort and fit for different sizes and body shapes of wearers, since some wearers have their largest dimensions over the hip area and other over the stomach. An improved adaptation of the belt dimension to the wearers body also when the wearer is moving is further achieved, since the belt may expand and retract in different parts along with the movements of the wearer.

The side panels or belt member 17, 18 having elastic regions 19, 20 therein can be manufactured in different ways. One way is to make cut-outs in a web material intended to form the side panels or belt members and to insert an elastic material in these cut-outs. An alternative way is to prepare a web of material having alternating inelastic and elastic regions extending the entire width of the web material and to divide this web of material in longitudinal direction and then displace the two web halves thus formed longitudinally with respect to each other, so that the desired configuration of alternating elastic and inelastic regions is formed, and subsequently join these two web halves to each other, for example by gluing, heat bonding or ultrasonic welding.

A further alternative is to use a web material that can be made elastic by activation, for example by being heated, and to activate only those parts of the web material which shall form the elastic regions.

The Elasticity test referred to above will now be described.

### Elasticity Test

The method measures the behaviour of an elastic material at repeated load and unload cycles. The sample is tested in the x-direction according to FIG. 1. The sample is stretched to a predetermined elongation and a cyclic movement between 0 and said predetermined elongation is performed. Desired load and unload forces are recorded. The permanent, i.e. remaining, elongation of the relaxed material is measured.

The permanent elongation after relaxation should be less than 10% and is measured by the method below. Thus an elastic elongation of 30% is defined as that the laminate should have a permanent relaxation after elongation of less than 10% after being exerted to an elongation of 30% in the tensile tester below. An elongation of 30% means an elongation to a length that is 30% longer than the initial length of the sample.

A tensile tester, e.g. Lloyd LRX™, able to perform cyclic movements and equipped with a printer/plotter or software presentation is used. The sample is prepared by cutting it to a width of 25 mm. The length and width of the sample may vary according to the available amount of material.

If the material to be tested has a width higher than 25 mm the sample should be cut so that the width is 25 mm. If however the material to be tested, e.g. the elastic material, has a width that is smaller than 25 mm the sample should have the width of the available material piece. The forces then have to be adjusted to the width of the sample according to the values given in brackets in the test method.

The tensile tester is calibrated according to the apparatus instructions. The parameters needed for the test (load and unload forces) are adjusted to:
Crosshead speed: 500 mm/min
Clamp distance: adapted to the length of the test sample
Preload: 0.05 N (0.02 N/10 mm width)

The sample is placed in the clamps and it is made sure that the sample is centred and fastened perpendicularly in the clamps. Depending on the length of the sample the distance between the clamps may vary. If a sample is longer than 100 mm it should be cut to a length of 100 mm. A suitable distance between the clamps is in this case 50 mm. For shorter samples the distance between the clamps can be shorter then 50 mm but as long as possible. For very short samples, less than 20 mm, the elastic sample should at both ends still be attached to inelastic material of the belt member or side panels, wherein the inelastic material is fastened in the clamps with the elastic part of the sample extending between the clamps. It is in this case important that the entire elastic part of such a sample is located between the clamps.

The tensile tester is started and two cycles between 0 and the predetermined elongation are performed. The crosshead should return immediately and not be held in the stretched position. There should not be any delay between the two cycles of the test method. After the last cycle, the sample is relaxed for 1 minute, then the permanent elongation is measured by stretching the sample until a force of 0.1 N (0.04 N/10 mm width) is detected and the elongation is read.

Although only a few exemplary embodiments have been described in detail above, those skilled in the art will readily understand that many modification are possible and that any such modifications are intended to be included within the scope of this invention, which is defined in the following claims.

1. An absorbent garment having a longitudinal direction and a lateral direction and comprises a first body panel, a second body panel and a crotch portion there between, each of said first and second body panels having a waist portion, said garment further being provided with a pair of side panels or at least one belt member extending laterally outwardly on either side of the waist portion of the second body panel and each side panel or belt member having a length in the lateral direction, wherein said side panels or at least one belt member each has an upper edge and a lower edge and a laterally extending centre line there between dividing each side panel or belt member in an upper part extending between said upper edge and said lateral centre line and a lower part extending between said lower edge and said lateral centre line, said side panels or the at least one belt member being either fixedly or releasably attached to the second body panel, wherein at least one of said side panels or the at least one belt members comprises an elastic member, while a rest of the side panel or belt member is of an inelastic material, said elastic member in at least one of said side panels or the at least one belt member comprises at least a first elastic region and a second elastic region, each extending over less than an entirety of the length of the respective side panel or the at least one belt member and over less than an entirety of the distance between the upper and lower edges of the respective side panel or the at least one belt member, wherein the first elastic region has a main part of its surface area on the upper part of the respective side panel or the at least one belt member, so as to allow elastic expansion of the upper edge in the area of the first elastic region, and there is a portion of inelastic material extending from the first elastic region to the lower edge of the side panel or the at least one belt member, and the second elastic region has a main part of its surface area on the lower part of the respective side panel or the at least one belt member, so as to allow elastic expansion of the lower edge in the area of the second elastic region, and there is a portion of inelastic material extending from the second elastic region to the upper edge of the side panel or the at least one belt member.

2. The absorbent garment according to claim 1, wherein said elastic regions extend over from 20 to 80% of the distance between the upper and lower edges of the respective side panel or belt member.

3. The absorbent garment according to claim 2, wherein said elastic regions each has an elastic region length in the lateral direction of the side panel or the at least one belt member and an elastic region height in a direction transverse thereto, wherein the height of each elastic region is at least 2 cm.

4. The absorbent garment according to claim 3, wherein said side panel or the at least one belt member has a second height in a direction transverse to the lateral direction of between 8 and 40 cm.

5. The absorbent garment according to claim 1, wherein said elastic regions each has an elastic region length in the lateral direction of the side panel or the at least one belt member of between 1.5 and 20 cm.

6. The absorbent garment according to claim 1, wherein at least one of said side panels or the at least one belt member comprises at least three elastic regions, a first elastic region, a second elastic region and a third elastic regions, wherein also the third elastic region extends over only a part of the length of the respective side panel or the at least one belt member and over only a part of the distance between the upper and lower edge of the respective side panel or the at least one belt member and wherein the third elastic region has a main part of its surface area on the upper or lower part of the respective side panel or the at least one belt member, so as to allow elastic expansion of the upper edge or lower edge, respectively, in the area of the third elastic region, and there is a portion of inelastic material also between the third elastic region and the lower or upper edge respectively of the side panel or the at least one belt member.

7. The absorbent garment according to claim 6, wherein the third elastic zone is located on the upper part of the respective side panel or the at least one belt member.

8. The absorbent garment according to claim 6, wherein at least one of said side panels or the at least one belt member comprises at least four elastic regions, a first elastic region, a second elastic region, a third elastic region and a fourth elastic regions, wherein also the fourth elastic region extends over only a part of the length of the respective side panel or the at least one belt member and over only a part of the distance between the upper and lower edge of the respective side panel or the at least one belt member and wherein the fourth elastic region has a main part of its surface area on the upper or lower part of the respective side panel or the at least one belt member, so as to allow elastic expansion of the upper edge or lower edge, respectively, in the area of the fourth elastic region, and there is a portion of inelastic material also between the fourth elastic region and the lower or upper edge respectively of the side panel or the at least one belt member.

9. The absorbent garment according to claim 8, wherein two elastic zones are located on the lower part and two elastic zones are located on the upper part of the respective side panel or the at least one belt member.

10. The absorbent garment according to claim 8, wherein at least one of said side panels or the at least one belt member comprises at least five elastic regions, each extending over only a part of the length of the respective side panel or the at least one belt member and over only a part of the distance between the upper and lower edge of the respective side panel or the at least one belt member, so as to allow elastic expansion of the upper edge or lower edge, respectively, in the area of each elastic region.

11. The absorbent garment according to claim 10, wherein at least three of said elastic regions are located on the upper part of the respective side panel or the at least one belt member.

12. The absorbent garment according to claim 1, wherein both side panels comprise said elastic regions.

13. The absorbent garment according to claim 1, wherein there are two belt members and the two belt members are adapted to be wrapped around the waist of the wearer of the garment and fastened together by means of a first fastener, the first body panel at its waist portion being provided with a second fastener adapted to be fastened to the belt members in such a way that the garment will assume a pant-like shape.

14. The absorbent garment according to claim 1, wherein said side panels are adapted to be fastened to the first body panel by means of a first fastener.

15. The absorbent garment according to claim 1, wherein the elastic regions comprise an elastic film or an elastic laminate.

16. The absorbent garment according to claim 1, wherein the elastic regions comprises one or more elastic threads or strips contractibly affixed between web materials.

17. The absorbent garment according to claim 1, wherein at least a substantial part of the side panels or the at least one belt member comprises a material that can be made elastic by activation and that the elastic regions are regions that have been activated so as to become elastic.

18. The absorbent garment according to claim 1, wherein the elastic regions of the respective side panel or belt member are provided with a contrasting color, pattern or marking so as to be distinguishable from the rest of the side panel or the at least one belt member, or alternatively that said rest of the side panel or the at least one belt member is provided with a contrasting color, pattern or marking so as to be distinguishable from said elastic regions.

19. The absorbent garment according to claim 1, wherein the absorbent garment is a diaper or an incontinence guard.
